SEQUENCE LISTING (14 sequences)

SEQ ID NO: 1 - DNA, 19 bp, Artificial Sequence
  Description of Artificial Sequence Synthetic primer
tggtgttggcctcacacct

SEQ ID NO: 2 - DNA, 20 bp, Artificial Sequence
  Description of Artificial Sequence Synthetic primer
ccaggagagaattgttgctc

SEQ ID NO: 3 - DNA, 21 bp, Artificial Sequence
  Description of Artificial Sequence Synthetic probe
ccacagcaccctcaaacctgc

SEQ ID NO: 4 - DNA, 18 bp, Artificial Sequence
  Description of Artificial Sequence Synthetic primer
gaaggtgaaggtcggagt

SEQ ID NO: 5 - DNA, 19 bp, Artificial Sequence
  Description of Artificial Sequence Synthetic primer
gaagatggtgatgggattt

SEQ ID NO: 6 - DNA, 19 bp, Artificial Sequence
  Description of Artificial Sequence Synthetic probe
caagcttcccgttctcagc

SEQ ID NO: 7 - DNA, 25 bp, Artificial Sequence
  Description of Artificial Sequence Synthetic primer
ccaccaaccaccaggctacaggggc

SEQ ID NO: 8 - DNA, 27 bp, Artificial Sequence
  Description of Artificial Sequence Synthetic primer
aggctcctcctttccaggtcagttagc

SEQ ID NO: 9 - DNA, 20 bp, Artificial Sequence
  Description of Artificial Sequence Synthetic primer
cagaggactagaacacctgc

SEQ ID NO: 10 - DNA, 19 bp, Artificial Sequence
  Description of Artificial Sequence Synthetic primer
gctggtgaaaaggacctct

SEQ ID NO: 11 - DNA, 17 bp, Artificial Sequence
  Description of Artificial Sequence Synthetic primer
ccacccgctcgcttctc

SEQ ID NO: 12 - DNA, 20 bp, Artificial Sequence
  Description of Artificial Sequence Synthetic primer
cactgacagcgcagctcatt

SEQ ID NO: 13 - DNA, 25 bp, Artificial Sequence
  Description of Artificial Sequence Synthetic primer
ccttgctgttcttctctgtaccatg

SEQ ID NO: 14 - DNA, 24 bp, Artificial Sequence
  Description of Artificial Sequence Synthetic primer
gcaatcagttccaggtgagtgatg

(19) United States
(12) Patent Application Publication
(10) Pub. No.: US 2016/0250327 A1
(43) Pub. Date: Sep. 1, 2016

(54) METHODS AND COMPOSITIONS TO REDUCE LIVER DAMAGE ASSOCIATED WITH CONDITIONS OR THERAPIES THAT AFFECT THE IMMUNE SYSTEM

(71) Applicant: THE UNIVERSITY OF CHICAGO, Chicago, IL (US)

(72) Inventor: Yang-Xin Fu, Chicago, IL (US)

(21) Appl. No.: 15/082,898

(22) Filed: Mar. 28, 2016

Related U.S. Application Data
(63) Continuation of application No. 13/183,617, filed on Jul. 15, 2011, now abandoned.
(60) Provisional application No. 61/365,243, filed on Jul. 16, 2010.

(51) Int. Cl. A61K 39/395; A61K 31/713

(52) U.S. Cl. CPC A61K 39/3955; A61K 31/713

(57) ABSTRACT

One side-effect arising from the use of antibodies against TNF receptor family members as therapeutics can be liver damage which precludes the completion of clinical trial. A novel LT-dependent pathway is described that mediates liver cell injury in several disease models.

### CROSS REFERENCE TO RELATED APPLICATIONS

This application is a Continuation of copending U.S. patent application Ser. No. 13/183,617, filed Jul. 15, 2011, which claims the benefit of priority from U.S. provisional application No. 61/365,243, filed Jul. 16, 2010. The above-referenced disclosures are herein incorporated by reference in their entireties.

This invention was made with government support under AI062026, CA115540 and DK58891 awarded by The National Institutes of Health. The government has certain rights in the invention.

### SEQUENCE LISTING

The instant application contains a Sequence Listing which has been submitted in ASCII format via EFS-Web and is hereby incorporated by reference in its entirety. Said ASCII copy, created on Jul. 13, 2011, is named 113458_SEQ_ST25.txt and is 3,475 bytes in size.

### BACKGROUND

Immune-mediated injury of the liver arises in diseases such as autoimmune hepatitis, primary biliary cirrhosis, and infectious viral hepatitis B and C. These diseases have few treatment options and cause significant morbidity and mortality in the US and worldwide. Concanavalin A (ConA)-induced hepatitis has been investigated as a mouse model of T cell-mediated liver injury; however, the exact pathway leading to hepatic injury is poorly defined and multiple pathways appear to exist. Understanding the etiology of immune-mediated liver cell injury will enable development of novel therapeutic targets, and compositions for preventing or ameliorating liver damage.

Following ConA administration, a variety of cellular and cytokine components contribute to liver injury. ConA induces the expression of multiple cytokines that regulate hepatocyte cell death and survival. Initial studies implicated CD4⁺ T cells in ConA provoked liver injury by noting that CD4 T cell depletion in mice was prophylactic. Later studies revealed that components of the innate immune system, such as neutrophils, Kupffer cells, eosinophils, as well as NKT cells also contribute to liver injury induced by ConA administration.

TNF superfamily members (TNFSF) play a central role in ConA-induced hepatitis. Among the earliest observations was an increase in serum TNF after ConA administration. The Fas/FasL system appears to be necessary for ConA hepatitis since gld/gld mice, in which FasL is defective, and lpr mice, in which Fas is defective, are reported resistant to ConA-induced liver injury. More recently the expression of FasL on NKT cells has been reported to be sufficient to mediate ConA-induced hepatitis.

Another TNF superfamily member CD137 (4-1BB) which is primarily expressed on activated T cells, can contribute to liver injury via production of proinflammatory cytokines such as TNF and IFN-γ. Increased IFN-γ, IL-12, IL-5, IL-27, or IL-4 cytokines in serum after ConA administration exacerbate toxicity to hepatocytes, and a genetic deficiency in any one of these cytokines yields mice resistant to ConA-mediated liver injury. In contrast, IL-6, IL-10, IL-15, and IL-22 cytokines are reported to provide protection from ConA-induced liver injury. Trafficking of leukocytes to the liver is another critical mechanism in ConA-mediated liver injury. Production of chemokines such as MIP-2 (CXCL2) or MIP-1α (CCL3) heighten liver injury by promoting migration of inflammatory cells into the liver.

Lymphotoxin (LT) is a member of the TNF superfamily of cytokines with pleiotropic functions in the immune system and has two known forms. Membrane-anchored LT, heterotrimeric LTα₁β₂, interacts exclusively with the Lymphotoxin β receptor (LTβR) while soluble LT, homotrimeric LTα₃, interacts with TNFRs I and II. Both TNFR and LTβR play a role in the organogenesis and maintenance of secondary lymphoid organs. Membrane LT expression is primarily restricted to lymphocytes and lymphoid tissue inducing cells; whereas LTβR is not expressed on lymphocytes, but by stromal and hematopoietic cells, including macrophages and DC. The contribution of specific LTβR expressing cells in hepatitis remains unknown.

Aside from membrane LTα₁β₂, LTβR interacts with another membrane ligand, LIGHT (“lymphotoxin-like, exhibits inducible expression and competes with herpesvirus glycoprotein D for HVEM, a receptor expressed by T lymphocytes”), also known as TNFSF14. While membrane LTα1β2 exclusively binds LTβR, LIGHT can also signal to herpes virus entry mediator (HVEM) expressed by distinct cell types. In contrast to LT, LIGHT is not essential for the form of lymphoid tissues, but has a potent T cell costimulatory function affecting CTL-mediated tumor rejection, intestinal inflammation, allograft rejection, liver regeneration, and graft versus host disease. Previous studies were focused on LIGHT and implicated the role of LIGHT signaling through both LTβR and HVEM in the pathogenesis of ConA-induced hepatitis by an undefined mechanism, whereas the contribution of LT was largely ignored.

### SUMMARY

A novel LT-dependent pathway is described that mediates liver cell injury in several disease models. Controlling LT signaling is an additional if not primary dimension of therapeutic intervention for a wide array of human liver diseases.

T cell activation is an early and critical event in liver injury mediated by various therapeutic antibodies, and conditions and diseases such as viral or autoimmune hepatitis. Multiple TNFSF cytokines activate critical pathways which are essential or sufficient for T cell-mediated hepatic injury. Observations in recent clinical trials of severe liver injury after the repeated use of agonistic antibodies against CD137 and LTβR, both TNF receptor family members, illustrates the limitation on clinical trials due to liver damage. These serious side effects prevented further trials.

Unexpectedly, the LTβR and TNFR pathways were found to be essential for the pathogenesis of liver injury mediated by multiple TNFSF cytokines, including Fas, CD137, lymphotoxin, and LIGHT. Genetic interruption of the LTβR pathway, specifically on hepatocytes, prevents such liver injury in mice Inhibition of LTβR or TNFR signaling effectively protected mice from liver injury induced by various insults to the liver. These results suggest that TNFR and LTβR signaling on liver tissues is an essential pathway for the development of various forms of hepatitis and is a therapeutic target.

Disclosures herein illuminate a broader role of TNFR and LTβR in the pathogenesis of liver injury caused by ConA, Fas, or CD137 stimulation. This creates a new dimension to understanding the control of hepatocyte survival and homeostasis because LTβR and TNFR signaling integrates several TNF superfamily cytokines with distinct roles in liver injury. Methods and compositions disclosed herein allow for a new approach, blocking components of the TNF or LT pathways, to control immune pathology within the liver.

### BRIEF DESCRIPTION OF THE DRAWINGS

FIG. 1A-1G. LTβR-deficient mice are resistant to Fas-induced liver injury. WT and LTβR^{-/-} mice were injected i.v. with 0.3 mg/kg of anti-Fas antibody (Jo2). This data is representative of two independent experiments. (A) FIG. 1A Mice survival kinetics. n-number of mice per group. (B) (FIG. 1B) Alanine aminotransferase (ALT) levels in serum at 3 h after Jo2 injection. Data are presented as mean±SD, n=4. (FIGS. 1C and 1D) Reduced neutrophils recruitment to the liver in LTβR^{-/-} mice at 2 h after Jo2 injection. (C) (FIG. 1C) Lymphoid cells were purified from the liver by PERCOLL® gradient and stained with anti-Gr-1 and anti-CD11b antibodies by flow cytometry. (D) FIG. 1D_Representative hematoxylin and eosin and anti-myeloperoxidase (MPO) staining of liver sections at 2 h after Jo2 or control rat-IgG injection. Bars: 50 μm. (E) FIG. 1E shows reduced_expression of CXCL2 and (FIG. 1F) of CCL3 and (FIG. 1G) of CXCLI chemokines in the liver of LTβR^{-/-} mice at 2 h after Jo2 injection measured by real-time PCR. Data shown have been normalized to HPRT. Data shown are means±SD, n=4.

FIG. 2A-2G. Inhibition of LTβR signaling prevents hepatitis in a Fas-independent liver injury model. FIG. 1A-1C_Fas-independent liver injury model was induced by agonistic anti-CD137 treatment. Fas^{-/-} mice were injected i.p. with 200 micrograms of anti-CD137 antibody or control rat IgG. (A) (FIG. 1A Kinetics of ALT levels in serum. (B) (FIG. 1B) Hematoxylin and eosin staining (upper panel) and TUNEL apoptosis staining of livers on day 7 after anti-CD137 treatment. (C) (FIG. 1C)CD137-induced hepatitis is dominated by CD8 T cell inflammation. Total cell numbers of T cell subsets, B, NKT and NK cells in the liver at day 7 after anti-CD137 treatment. (D) (FIG. 1D) CD4⁺ T cells are not required for CD137-induced hepatitis. Hematoxylin and eosin staining of livers from Fas^{-/-} and Fas^{-/-}CD4^{-/-} mice on day 7 after anti-CD137 treatment. (E) (FIG. 1E) CD8⁺ T cells are essential for CD137-induced hepatitis. Hematoxylin and eosin (upper panel) or TUNEL staining for apoptosis (lower panel) of livers from Fas^{-/-} and Fas^{-/-} CD8^{-/-} mice on day 7 after anti-CD137 treatment. (FIG. 1F AND 1G) LTβR-Ig treatment prevents hepatitis in induced by agonistic anti-CD137 treatment. Fas^{-/-} mice were injected i.p. with 200 μg of anti-CD137 antibody or control rat IgG. LTβR-Ig (200 μg) was injected i.p. on day 0 and day 2 after anti-CD137 administration and serum ALT levels (F) (FIG. 1F), and hematoxylin and eosin liver sections (G) analyzed on day 7. Comparable results were obtained in two independent experiments. Data shown in (FIGS. 1A, 1C and 1F) represent means±SD, n=4 mice per group. Bars: 100 μm.

FIG. 3A-3G. Fas-independent liver injury model induced by agonistic anti-CD137 treatment. (A) (FIG. 3A) Fas^{-/-} mice were injected i.p. with 200 μg of anti-CD137 antibody or control rat-IgG and total leukocyte cell numbers in the liver analyzed on day 7 and 14. (FIGS. 3B and 3C) Cytokine expression in liver after agonistic anti-CD137 treatment. (B) (FIG. 3B) Agonistic anti-CD137 treatment induces IFN-γ production by T cells. Fas^{-/-} mice were injected i.p. with 200 μg of anti-CD137 antibody or control rat-IgG and IFN-γ expression by T cells was analyzed by flow cytometry at day 7 after treatment. (C) (FIG. 3C, 3D) Agonistic anti-CD137 treatment increases production of proinflammatory cytokines in the liver. Fas^{-/-} mice were injected i.p. with 200 μg of anti-CD137 antibody or control rat-IgG. Cytokine production in the liver at 7 days after treatment was measured by using cytokine bead assay (BD Biosciences). (FIGS. 3E and 3F) ALT levels in serum of livers from Fas^{-/-} and Fas^{-/-}CD4^{-/-} mice (D), or Fas^{-/-} and Fas^{-/-}CD8^{-/-} mice (E) (FIG. 3F) on day 7 after anti-CD137 treatment. (F) (FIG. 3G) LTβR-Ig treatment prevents hepatitis in induced by agonistic anti-CD137 treatment. Fas^{-/-} mice were injected i.p. with 200 μg of anti-CD137 antibody or control rat-IgG. LTβR-Ig (200 μg) was injected i.p. on day 0 and day 2 after anti-CD137 administration and leukocyte cell number in the liver analyzed on day 7. Data shown are means±SD, n=3. Comparable results were obtained in two independent experiments.

FIGS. 4A and 4B. Fas-independent liver injury model induced by agonistic anti-CD137 treatment can be controlled by soluble TNFR or LTβR. (A) (FIG. 4A) Fas^{-/-} mice were injected i.p. with 200 μg of anti-CD137 antibody or control rat-IgG. Low dose of TNFR-Ig, LTβR-Ig (100 ug) or anti-IFNγ was used on day 0 after anti-CD137. Serum ALT and AST were monitored on day 7 after anti-CD137. (B) FIG. 4B. Fas^{-/-} mice were injected i.p. with 200 μg of anti-CD137 antibody or control rat-IgG. Low dose of anti-LTb antibody, LTbR-Ig (100 μg), HVEM-Ig, or anti-IFNγ was used on day 0 after anti-CD137. Serum ALT and AST were monitored on day 7 after anti-CD137.

FIGS. 5 and 5B. LTβR signaling by LT is essential for pathogenesis of hepatitis. (A) FIG. 1A. LTβ^{-/-} and LT βR^{-/-} deficient mice are resistant to ConA-induced hepatitis. WT, LTβ^{-/-}, LT βR^{-/-}, and TNFR^{-/-} mice received a sublethal dose of ConA (13 mg/kg). ALT levels were measured 24 h later. Data shown are means±SD. n=5 mice per group. Comparable results were obtained in three independent experiments. (B) (FIG. 1B). Liver sections from indicated mice sacrificed 24 h after ConA injection were stained with hematoxylin and eosin. ConA treatment induces geographic necrosis in WT mice with minimal liver necrosis in LTβ^{-/-} and LTβR^{-/-} mice. All histology displayed at original magnification. Histology pictures indicative of results from one of three independent experiments.

FIG. 6A-6D. LTβR-Ig fusion protein treatment protected from ConA-induced liver injury. (FIG. 6A-6C). WT mice (n=5) received 200 μg i.v. LTβR-Ig or control human IgG two hours prior to 20 mg/kg of ConA injection. (A) (FIG. 6A) Hematoxylin and eosin staining of liver sections obtained 24 hours post ConA injection. (B) (FIG. 6B) Serum collected at 24 hours assayed for ALT activity. (C) (FIG. 6C) TUNEL liver section staining for apoptosis at 8 hours following ConA injection. (D) (FIG. 6D) LT blockade via anti-LTβ antibody reduces ConA-induced liver injury. WT mice received an i.v. lethal dose of ConA (20 mg/kg) and either pretreated with hamster anti-LTβ antibody (50 μg) (n=5 mice per group) or control hamster HA4/8 antibody (n=5) two hours prior to ConA injection. Serum was assayed at 24 h for ALT activity. Represents one of two independent experiments.

FIG. 7. LT expression is increased in livers of hepatitis C patients. LTα mRNA expression in the livers of normal donors and hepatitis C patients was measured by real-time PCR. Statistical significance was determined by a two-tailed unpaired Student's t-test (**p=0.0027).

### DETAILED DESCRIPTION

Activated T cell-induced liver injury in viral and autoimmune hepatitis involves multiple TNFSF members and various signaling pathways. Tempering such injury by a single treatment has been difficult. Relieving the staggering worldwide burden of liver diseases requires new understanding of pathogenesis and new treatments. The multiple dimensions of LTβR (and TNFR) signaling revealed its focal responsibility for integrating distinct hepatocyte injury signaling pathways. The dependence of distinct hepatitis models on LTβR and TNFR signaling was shown; murine hepatitis was induced by stimulation of TNFSF members Fas, CD137, LIGHT, or even ConA, a general T cell activator. Surprisingly, use of genetic knockout and soluble protein systems revealed that relevant LTβR signaling in ConA pathogenesis depends on membrane LT complex signaling directly on hepatocytes. TNFR and LTβR-dependent pathogenic mechanisms involve both the induction of hepatocyte apoptosis and the regulation of inflammation by neutrophil recruitment. A single treatment with soluble LTβR fusion protein (LTβR-Ig) blocked liver injury from a variety of insults encompassing multiple signaling pathways. These new insights open a new dimension for achieving dynamic control of hepatic immune pathology with treatment implications for hepatitis and multiple forms of liver injury.

A recent study using a monoclonal antibody that limited LIGHT binding to LTβR, while minimally affecting the binding of LT, suggested an independent role of LIGHT in the pathogenesis of hepatitis, presumably acting through LTβR (Anand et al., 2006). In contrast, an independent study suggested LIGHT works through HVEM signaling in ConA-induced hepatitis (An et al., 2006). However, HVEM-deficient mice show a greater susceptibility to ConA-induced inflammation that calls into question the importance of HVEM signaling in LIGHT-mediated hepatitis (Wang et al., 2005). LTβR signaling from the surface LT ligand has a non-redundant role in the development of hepatitis. Furthermore, analysis of conditional LTβR-deficient mice was interpreted to mean that LTβR expression specifically on hepatocytes is essential for ConA-induced liver injury.

LT-LTβR-dependent pathogenic mechanism likely involves both regulation of inflammation by neutrophil recruitment and induction of hepatocyte apoptosis. The role of LTβR in neutrophil recruitment is novel and intriguing since no defect in neutrophil development is reported in LT- or LTβR-deficient mice. Gene microarray analysis revealed that expression of several neutrophil specific genes, such as myeloperoxidase and lactoferrin, were reduced in LTα^{-/-} spleen compared to WT mice (Shakhov et al., 2000). Reduced numbers of neutrophils in the livers of LTβR-deficient mice following ConA administration compared to WT mice and increased numbers when LTβR was stimulated with adenovirus-delivered LIGHT have been observed. CXCL2 are CCL3 are potent chemokines controlling neutrophil recruitment to the liver (Nakamura et al., 2001; Ramos et al., 2006; Bajt et al., 2001; Rawaiah et al., 2007). Previous studies showed that blocking CXCL2 or CCL3 with specific antibodies or inhibitors effectively protected mice from ConA-induced hepatitis (Ajuebar et al., 2004; Okamoto et al., 2005). The present disclosure supports that LTβR signaling regulates CXCL2 and CCL3 expression thus promoting neutrophil recruitment to the liver after ConA administration.

How LTβR signaling promotes hepatocyte apoptosis is not entirely clear. In contrast to TNFRI or Fas receptors, LTβR lacks the death effector domains known to trigger caspase activation and apoptosis (Ware, 2005). However, there is evidence that LIGHT-induced LTβR signaling can rapidly recruit TRAF3, TRAF2, cIAP1, and Smac adaptors (Kuai et al., 2003; Kim et al., 2005). TRAF3 activation via LTβR signaling appears to induce apoptosis in some adenocarcinoma cell lines, whereas TRAF2 signaling promotes NF-κB and JNK activation (Kim et al. 2005). Several recent studies suggest that TRAF3 inhibits the noncanonical NF-κB activation pathway by suppressing p100 processing through induction of NIK degradation. Another report shows pretreatment of human primary hepatocytes with LIGHT induces NF-κB activation and blocks apoptosis induced by TNF/Actinomycin-D but not Fas-induced apoptosis. Cell context and balance between NF-kB and apoptotic pathways all appear to influence cell fate. Furthermore, LTβR^{-/-} mice were protected from Fas and ConA-induced liver injury, suggesting collaboration of LTβR and Fas signaling pathways leading to liver injury. LTβR-dependent up-regulation of neutrophil recruiting CXCL2 and CCL3 chemokines after Fas and ConA stimulation promotes liver injury. In line with this, both CXCL2 and CCL3 are known to be transcriptionally activated by NF-kB signaling, which is induced following Fas and LTβR stimulation.

In contrast to a hepatitis model, previous work by the inventors demonstrated that LTβR signaling promotes liver regeneration after partial hepatectomy. These similar seemingly paradoxical relationships exist for other members of the TNF superfamily of cytokines, Fas and TNF for instance. Injection of Fas agonistic antibody induced massive liver injury in WT, but not in TNFRI/II^{-/-} mice, while the same treatment promoted liver regeneration after partial hepatectomy. LTβR signaling may be constantly maintained by LT and such tuning will coordinate with other TNFRSF members to control homeostasis of liver regeneration and injury. TNF, IFN-γ, and FasL are all induced after ConA administration. Therefore, it is possible that stimulation of hepatocyte LTβR by LT-expressing T and NKT cells causes apoptosis depending upon the presence of inflammatory cytokines such as TNF, IFNγ, FasL. Therefore, inhibitors of TNF or LT, such as soluble receptors for TNF or LT, could be used as a treatment to reduce liver injuries by rather broad insults.

An integrative role of LTβR (and TNFR) likely exists in connecting various pathways that control hepatocyte apoptosis and survival. Understanding the mediators of T cell-mediated liver injury provides targets for difficult-to-treat human liver diseases such as autoimmune and viral hepatitis Inhibition of LTβR signaling is effective in preventing both acute ConA-induced fulminant hepatitis and a subacute hepatitis induced by CD137 stimulation in Fas-deficient mice. In contrast, experimental approaches using stimulation of LTβR signaling are being pursued to improve liver regeneration, or anti-tumor therapy. Selective elimination of LTβR “side effects”, such as neutrophil-induced liver inflammation may improve efficacy of these treatments. This could also be achieved through inhibition of CXCL2 and CLL3 activity using neutralizing antibodies.

Current therapeutics in development that may result in hepatic liver damage that could be alleviated by inhibition or antagonism of the TNF and/or LT pathways include BMS-663513, a CD137 agonistic antibody. Other antibody therapies that may result in liver damage that could be alleviated by inhibition or antagonism of the TNF and or LT pathways include those that target coinhibitory and costimulatory T cell receptors. Coinhibitory and costimulatory molecules include cytotoxic T-lymphocyte antigen-4 (CTLA-4), glucocorticoid-induced tumor necrosis factor family receptor (GITR), B7-H1, programmed death [PD]-1, B7-H3 and B7x.

Agonistic antibodies for CD137 are being developed for the treatment of cancer, autoimmune and other immune related diseases, viral disease, by enhancing the response to vaccines and alleviating inflammatory disease.

Cancer broadly refers to cellular-proliferation and/or cellular growth disease states. The cancer may be breast, prostate, ovarian, brain, melanoma, colorectal, liver, lymphoma, lung, oral, head, neck, spleen, lymph node, small intestine, large intestine, blood cells, stomach, pancreatic, endometrium, testicle, skin, esophagus, bone marrow, blood, cervical, bladder, Ewing's sarcoma, thyroid, a glioma, and/or gastrointestinal cancers. Cancer also includes but is not limited to: sarcoma, myxoma, rhabdomyoma, fibroma, lipoma and teratoma, bronchogenic carcinoma, alveolar (bronchiolar) carcinoma, bronchial adenoma, chondromatous hamartoma, mesothelioma, squamous cell carcinoma, colorectal adenocarcinoma, leiomyosarcoma, carcinoma of the stomach, pancreatic ductal adenocarcinoma, insulinoma, glucagonoma, gastrinoma, pancreatic carcinoid tumors, vipoma, cancers of the small bowel cancers of the large bowel, colorectal adenocarcinoma, kidney adenocarcinoma, Wilm's tumor, nephroblastoma, bladder and urethra carcinomas, prostate adenocarcinoma and sarcoma, testicular cancers, hepatoma, hepatocellular carcinoma, cholangiocarcinoma, hepatoblastoma, angiosarcoma, hepatocellular adenoma, hemangioma, osteosarcoma, fibrosarcoma, malignant fibrous histiocytoma, chondrosarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma and giant cell tumors; granuloma, xanthoma, osteitis defornians, meningioma, meningiosarcoma, gliomatosis, astrocytoma, medulloblastoma, glioma, ependymoma, germinoma; pinealoma, glioblastoma multiformae, oligodendroglioma, schwannoma, retinoblastoma, congenital tumors, neurofibroma, breast cancer, endometrial carcinoma, cervical carcinoma, pre-tumor cervical dysplasia, ovarian carcinoma; serous cystadenocarcinoma, mucinous cystadenocarcinoma, unclassified carcinoma; granulosa-theca cell tumors, Sertoli Leydig cell tumors, dysgerminoma, malignant teratoma, vulval cancer, vaginal cancer fallopian tube carcinoma, chronic and acute myeloid leukemia, acute lymphoblastic leukemia, chronic lymphocytic leukemia, myeloproliferative diseases, multiple myeloma, myelodysplastic syndrome, Hodgkin's disease, non-Hodgkin's lymphoma, malignant lymphoma, endothelioma, malignant melanoma, basal cell carcinoma, squamous cell carcinoma, Kaposi's sarcoma, moles dysplastic nevi, lipoma, angioma, dermatofibroma, keloids, psoriasis, and neuroblastoma. The invention is applicable to other cancers discussed herein, including pre-cancers.

Immune related diseases include systemic lupus erythematosis, rheumatoid arthritis, osteoarthritis, juvenile chronic arthritis, spondyloarthropathies, systemic sclerosis, idiopathic inflammatory myopathies, Sjögren's syndrome, systemic vasculitis, sarcoidosis, autoimmune hemolytic anemia, autoimmune thrombocytopenia, thyroiditis, diabetes mellitus, immune-mediated renal disease, demyelinating diseases of the central and peripheral nervous systems such as multiple sclerosis, idiopathic demyelinating polyneuropathy or Guillain-Barré syndrome, and chronic inflammatory demyelinating polyneuropathy, hepatobiliary diseases such as infectious, autoimmune chronic active hepatitis, primary biliary cirrhosis, granulomatous hepatitis, and sclerosing cholangitis, inflammatory bowel disease, gluten-sensitive enteropathy, and Whipple's disease, autoimmune or immune-mediated skin diseases including bullous skin diseases, erythema multiforme and contact dermatitis, psoriasis, allergic diseases such as asthma, allergic rhinitis, atopic dermatitis, food hypersensitivity and urticaria, immunologic diseases of the lung such as eosinophilic pneumonias, idiopathic pulmonary fibrosis and hypersensitivity pneumonitis, transplantation associated diseases including graft rejection and graft-versus-host-disease.

In addition to soluble receptors for TNF, a wide variety of inhibitors or antagonists of TNF are contemplated in this patent for reducing liver injury induced by various agents, including CD137. These TNF inhibitors include Infliximab (RemicadeA®), mouse-human chimeric anti-huTNF mAb; D2E7 (Humira™), fully human anti-huTNF mAb; Etanercept (Enbrel®), p75sTNF-RII-Fc (dimeric); PEG-p55sTNF-RI (monomeric); Lenercept, p55sTNF-RI-IgG1 (dimeric), CDP571 (CDR-grafted anti TNF ab) and CDP870/certolizumab (PEG-linked anti TNF Fab). Other inhibitors of TNF include small molecules (e.g., those described by He et al., 2005 Science 310:1022-1025; Haraguchi et al., AIDS Res Ther. 2006; 3: 8. Published online 2006 Mar. 31. doi: 10.1186/1742-6405-3-8; Strachan et al., 2000 J. Immunol. 164: 6560-6565; and U.S. patent application Ser. No. 10/833,871 entitled “Preparation of hymenialdisine derivatives and use thereof.” Other TNF inhibitory antibodies include those described in U.S. Pat. No. 7,160,542; U.S. patent application Ser. Nos. 10/043,436, and 11/180,219 Inhibitors of TNF, inhibitors of TNF include soluble TNF receptor polypeptides or inhibitors of TNF receptors. Other inhibitors include competitive TNF antagonists, including those described in U.S. Pat. No. 5,795,967. Other inhibitors include nucleic acids such as siRNAs that are specific for inhibiting TNF expression.

In addition to soluble receptors for LT, a wide variety of inhibitors or antagonists of LT are contemplated in this patent for reducing liver injury induced by various agents, including CD137. Inhibitors of LT include soluble forms of the LTβR extracellular domain. This can be achieved, for example, by fusing the extracellular domain of LTβR to an immunoglobulin constant heavy chain domain or to a human IgG Fc domain. Other inhibitors include antibodies that bind to LTβR or LT. Competitive antagonists of LT and nucleic acids inhibitors such as siRNAs that are specific for inhibiting LT expression are also contemplated.

### Example 1

### LTβR-Deficient Mice are Resistant to Fas-Induced Liver Injury

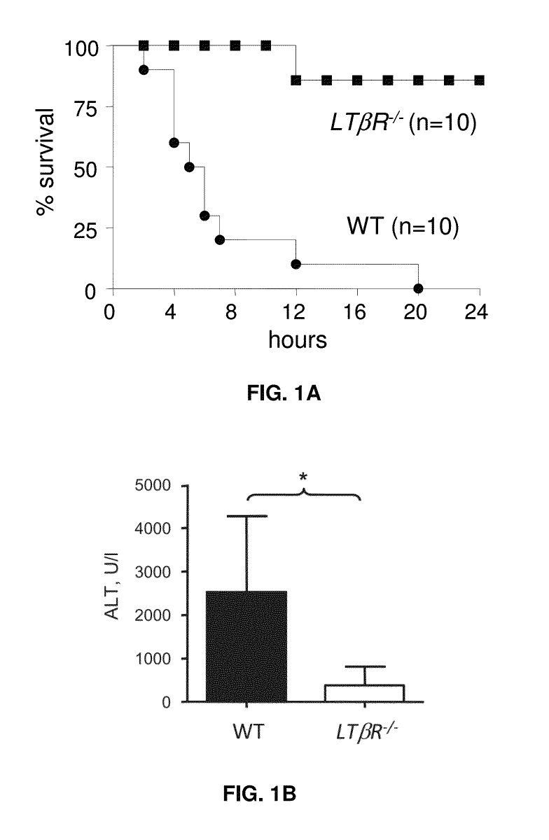

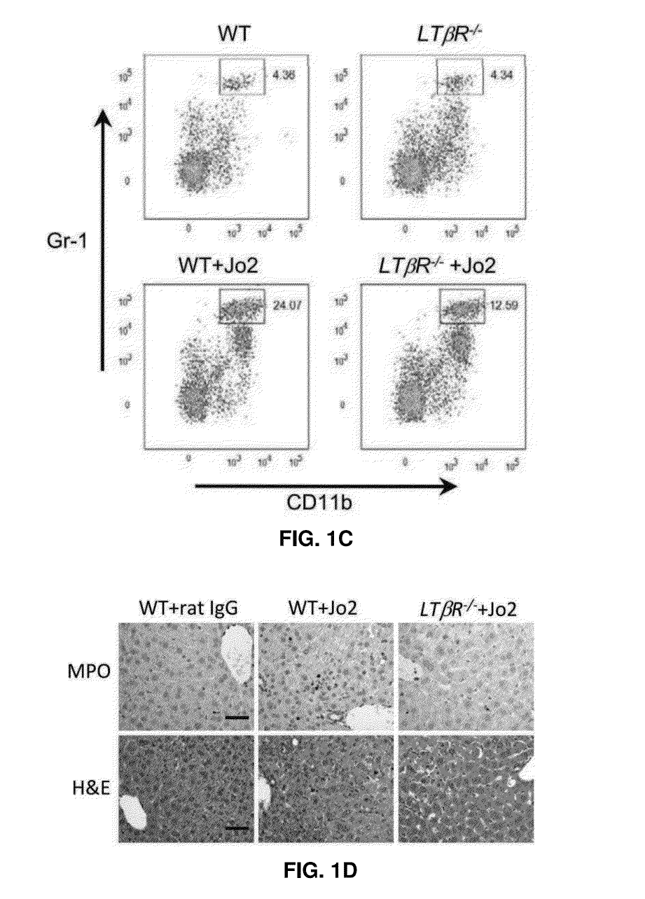

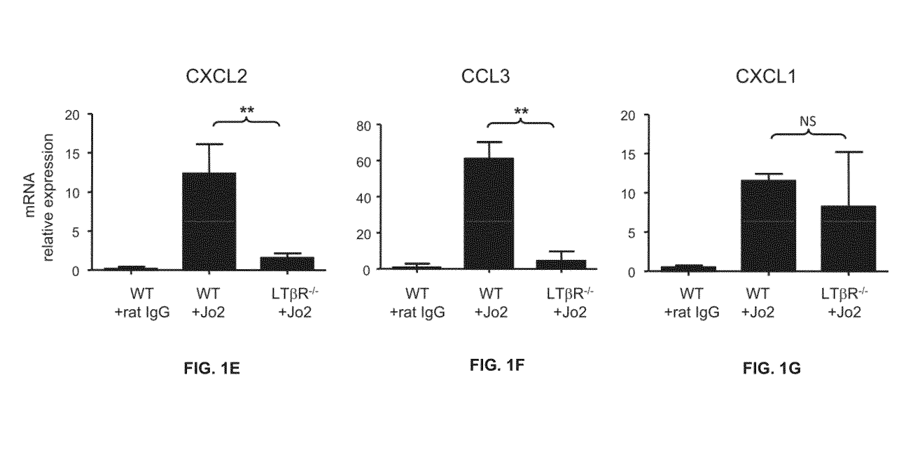

LTβR is credited with developing lymphoid tissues but its role in other tissues has not been fully explored. LTβR's high expression in liver and LTβR-deficient mice exhibiting reduced survival after partial hepatectomy suggest an active role of LTβR in promotion of hepatocyte proliferation or prevention of hepatic apoptosis. A question explored was whether LTβR signaling influences the liver injury induced by various TNFSF members. First, to investigate whether Fas-induced hepatocyte injury is dependent upon the LTβR signaling, WT and LTβR-deficient mice were injected with anti-Fas agonistic antibody (FIG. 1A-1G). This treatment induces severe hepatitis in wild type (WT) mice by directly targeting hepatocytes, leading to super-acute liver failure.

Unexpectedly, almost all LTβR^{-/-} mice survived, whereas WT mice died within 12 h after treatment (FIG. 1 A). Serum alanine aminotransferase (ALT) levels, as an indicator of liver injury, were significantly reduced in LTβR mice at 3 h after anti-Fas treatment (FIG. 1 B). Reduced liver injury in LTβR^{-/-} mice was not due to an intrinsic resistance of LTβR^{-/-} hepatocytes to Fas-mediated apoptosis because LTβR^{-/-} hepatocytes were sensitive to Fas-mediated apoptosis in primary hepatocytes in vitro.

To define which cells could influence Fas-induced hepatitis, liver lymphoid cells were analyzed by flow cytometry. Increased numbers of neutrophils were observed in WT mice injected with anti-Fas antibody (FIG. 1 C). However, neutrophil numbers were significantly reduced in LTβR^{-/-} mice, which correlated with reduced myeloperoxidase (MPO) positive cells and liver injury compared to WT mice at 2 h after anti-Fas antibody injection (FIGS. 1, C and D). The reduced recruitment of neutrophils to the liver of LTβR^{-/-} mice after Fas stimulation was not attributable to defective neutrophil development since nave LTβR^{-/-} mice contained normal neutrophil numbers in the liver and spleen (FIG. 1 C).

To define how LTβR controls neutrophil recruitment to the liver after Fas stimulation, expression of chemokines CXCL2, CXCL1, and CCL3 that control neutrophil recruitment to the liver was analyzed (FIG. 1E, FIG. 1F, FIG. 1G). CXCL2 and CXCL1 are rapidly induced in the liver after Fas stimulation and significantly contribute to neutrophil-mediated inflammation in the liver. Reduced expression of CXCL2 (MIP-2) and CCL3 was found in the liver of LTβR^{-/-} mice, while expression of CXCL1 (KC) was not significantly different between WT and LTβR^{-/-} mice (FIG. 1 E). These results support that LTβR promotes pathogenic recruitment of neutrophils to the liver after Fas stimulation by controlling expression of neutrophil attracting chemokines. Fas and LTβR signaling are linked and likely coordinate since Fas activation cannot fully induce liver injury in the absence of LTβR signaling.

### Example 2

### Inhibition of LTβR Signaling Prevents Hepatitis in a Novel Fas-Independent Liver Injury Model

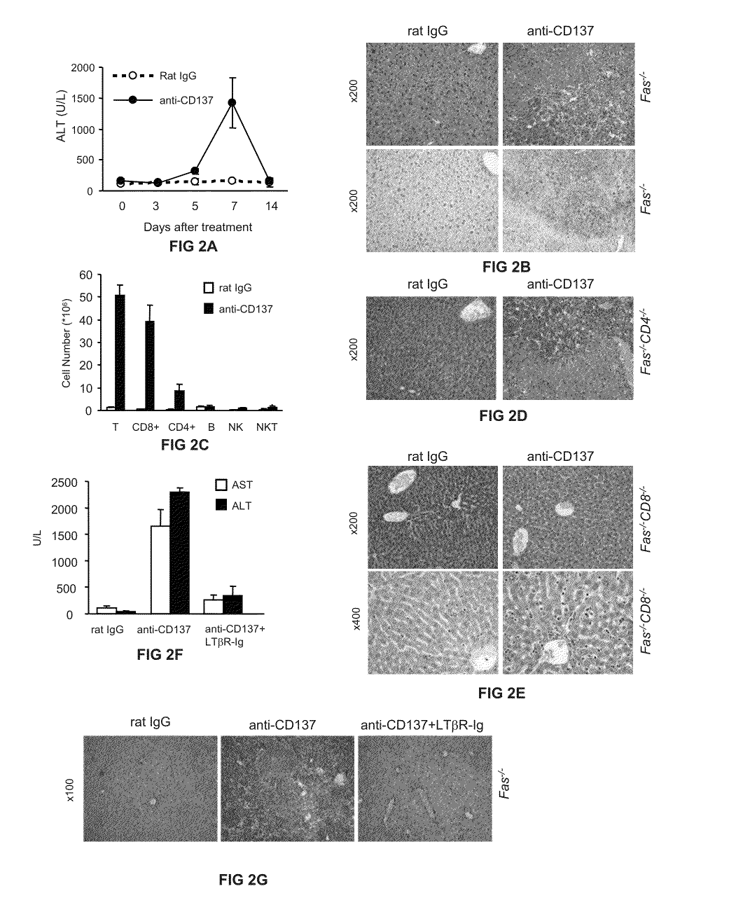

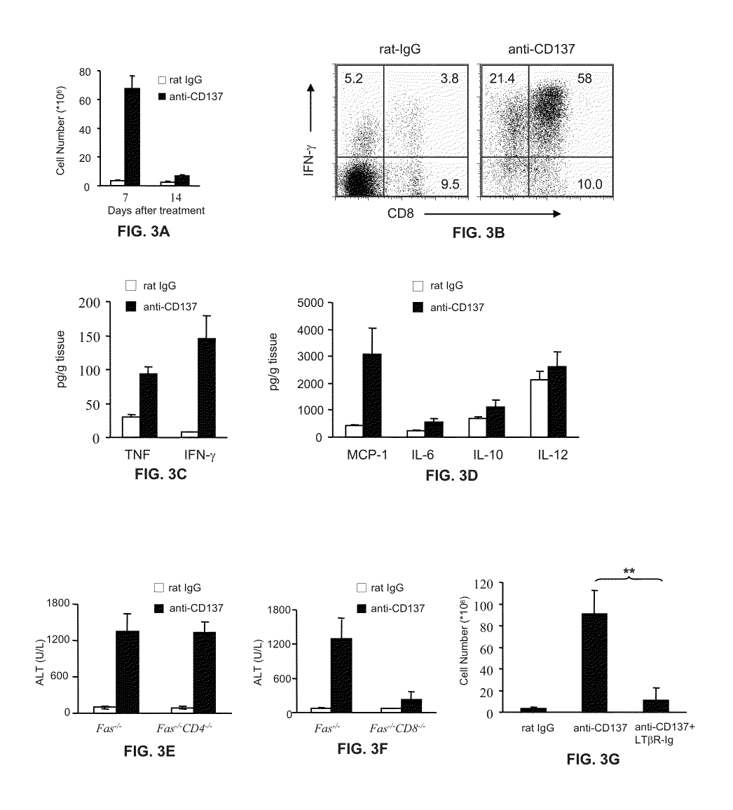

To define whether LTβR signaling is also involved in regulation of Fas-independent liver injury agonistic anti-CD137 antibody, a strong T cell activator, was injected into Fas^{-/-} mice. CD137 (4-1BB) is a member of the TNFR superfamily and primarily expressed on activated T cells. In WT mice this agonistic antibody induces subacute hepatitis within 7 days after injection that abates by day 14 (FIGS. 2, A and B). This detailed analysis of intrahepatic lymphocytes in this severe hepatitis model parallels viral hepatitis in humans, in which there is heavy infiltration of CD8⁺ cells that secrete large amounts of IFN-γ, TNF, and chemokines are observed (FIG. 2C, and FIGS. 3, A and 3C).

CD137-induced hepatitis appears to be rich in CD8+ T cells, which represented the largest cell population in the liver of treated mice, whereas CD4⁺ T cells showed a lesser increase (FIG. 2 C). However, unlike in ConA-induced hepatitis model (3), CD4 cells do not appear to play an essential role, since Fas^{-/-}CD4^{-/-} double deficient mice were susceptible to CD137-induced liver injury (FIG. 2 D and FIG. 3E). In contrast, Fas^{-/-}CD8^{-/-} mice were essentially resistant to CD137-induced liver injury (FIG. 2E and FIG. 3F). Thus, activation of T cells by the CD137 costimulation pathway in Fas-deficient mice results in development of a subacute CD8-dependent hepatitis in mice.

To test if inhibition of LTβR signaling could prevent hepatitis in the CD137-induced hepatitis and thus uncover a potential treatment, the effect of soluble LTβR fusion protein (LTβR-Ig) was tested. LTβR-Ig given immediately before and again two days after anti-CD 137 administration resulted in markedly attenuated hepatitis with reduced serum alanine ALT levels and minimal histological evidence of liver injury in Fas^{-/-} recipient mice (FIG. 2, F-G). LTβR-Ig treatment also reduced the number of infiltrating lymphocytes in the liver compared to control rat-IgG treated mice (FIG. 3G) Inhibition of LTβR signaling by injection of LTβR-Ig prevents the development of CD8-dependent, Fas-independent liver injury in CD137 stimulated mice.

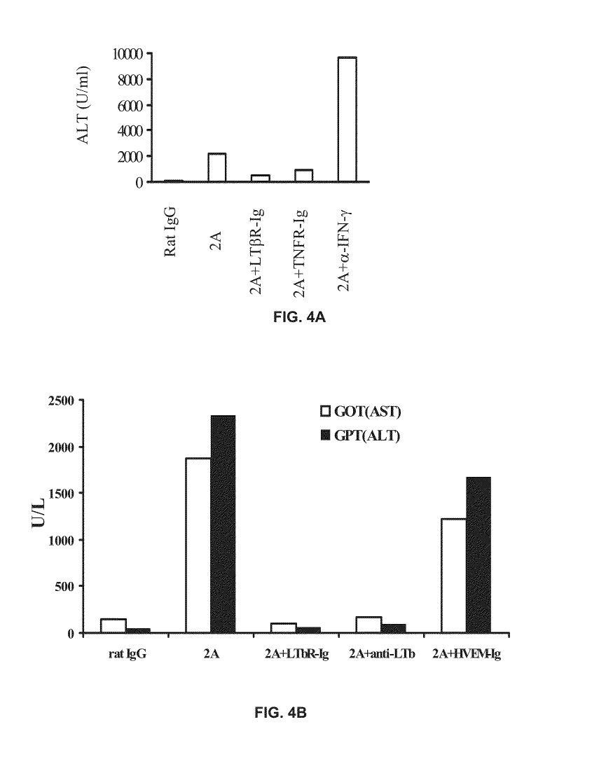

To test whether TNF is also involved in such T cell mediated hepatitis, the mice were treated with anti-CD137 as before, but 100 ug of TNFR-Ig and LTβR-Ig (FIG. 4A) was added. Interestingly, both TNFR-Ig and LTβR-Ig can greatly reduce ALT, supporting that TNF and LT are involved in liver injury. To test the role of LIGHT, another ligand for LTβR, HVEM-Ig that binds to LIGHT was used, but the blocking effect is limited (FIG. 4B). Therefore, it is likely that both LT and TNF are required, but not LIGHT.

### Example 3

### LTβR Signaling by Membrane LT Complex is Essential for ConA-Induced Liver Injury

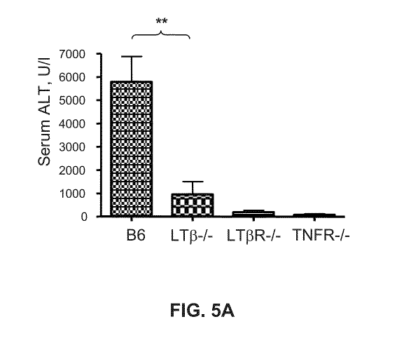

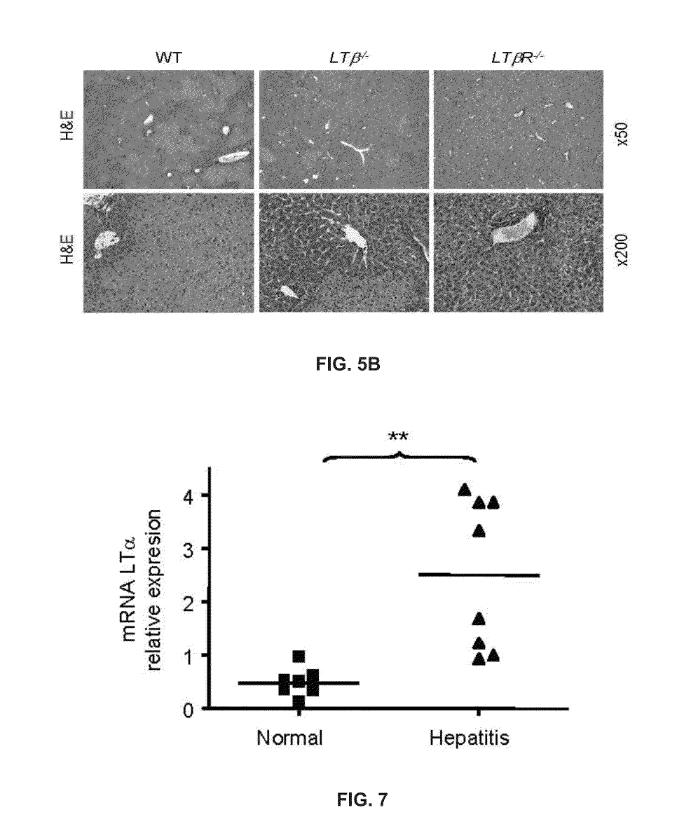

To determine whether LTβR signaling is required for liver injury induced by ConA, a strong and broader T cell activator that utilizes distinct ligands and cytokines that may each directly and indirectly cause liver injury, WT, LTβ^{-/-}, LTβR^{-/-}, and TNFR^{-/-} mice were injected with a sublethal dose of ConA (FIGS. 5 and 5B).

Quantitative serum ALT at 24 hours after ConA injection indicated significantly less hepatocyte injury in LTβ^{-/-}, and LTβR^{-/-} mice compared to WT mice (FIG. 5A). Examination of liver tissue harvested 24 hours after ConA administration revealed WT mice with geographic coagulative hepatocellular necrosis not seen in LTβ^{-/-} and LTβR^{-/-} mice (FIG. 5B). Residual liver injury present in LTβ^{-/-} compared to LTβR^{-/-} mice (FIG. 5A-B) suggests that LIGHT signaling, the only other known ligand for LTβR, may additionally contribute to liver injury. This data demonstrates a distinct role of LT specifically in the pathogenesis of hepatitis that is not redundant. Using a genetic knockout system, the inventor found that LTβ^{-/-} mice, lacking surface LT, were protected from ConA-induced liver injury even though unaltered LIGHT signaling persists (FIG. 5A-5B). Thus, this data demonstrates that LTβR deficiency confers resistance to ConA-induced liver damage and that signaling by the LTα₁β₂ through LTβR is necessary for developing ConA-induced liver injury. Therefore, blockade of LT or TNF by soluble receptors, or other inhibitors of LT or TNF expression or activity, will have broad impact on controlling liver injuries by various causes.

### Example 4

### Inhibition of LT and LTβR Signaling Protects Mice from ConA-Induced Liver Injury

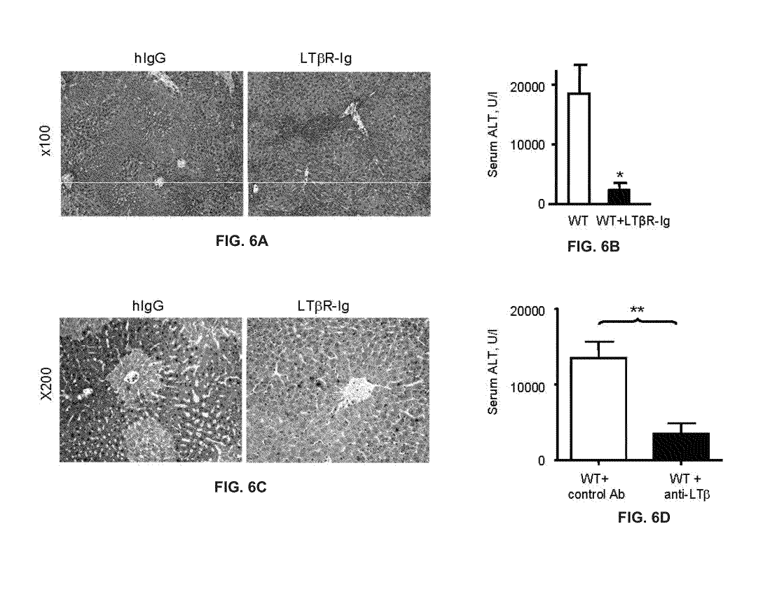

To determine whether inhibition of LTβR signaling could potentially protect mice from a broader form of liver injury induced by ConA, WT mice were pretreated i.v. with 200 μg of control human IgG or soluble LTβR fusion protein (LTβR-Ig), which blocks LTβR signaling, 2 hours prior to ConA injection (FIG. 6A-6D). Control human IgG i.v. 2 hours prior to ConA treatment in WT mice resulted in the expected marked ALT elevations at 8 and 24 hours post-injection, whereas LTβR-Ig resulted in substantially reduced ALT levels (FIG. 6B). The LTβR-Ig treatment markedly limited hepatocellular injury (FIG. 6A-6B). To determine the influence on liver apoptosis, livers were harvested at 8 hours post ConA administration and histological sections were assessed for TUNEL positive hepatocyte nuclei. WT mice treated with LTβR-Ig had significantly fewer TUNEL positive nuclei (FIG. 6C). These data indicate that a pharmacologic blockade of LTβR reduces ConA-induced hepatocyte injury, raising the possibility of treating broader forms of liver insults.

Since LTβR-Ig blocks signaling through the LTβR from both LIGHT and the membrane LT complex LTα1β2, the next test was whether specific inhibition of membrane LT reduces ConA-induced hepatocyte injury (FIG. 6D). Anti-LTβ antagonistic antibody given to WT mice 2 h prior to ConA administration reduced liver injury relative to treatment with a control hamster antibody (FIG. 6D). Together these data show that inhibition of LTβR signaling through LTα1β2 prevents ConA-induced liver injury and illuminate a new direction for treatment of hepatitis through blockade of LT or TNF by soluble receptors, or other inhibitors of LT or TNF expression or activity.

### Example 5

### LT Expression is Increased in Livers of Hepatitis C Patients

The LTβR pathway indeed plays a central and integral role in mediating the injury induced by different TNFSF members. This insight prompted the investigation of LT in T cell-mediated hepatitis, wherein multiple TNFSF pathways appear to coordinate the attack. Chronic hepatitis C (HCV) viral infection induces T cell-mediated hepatitis, a chronic inflammation associated with an increased production of inflammatory cytokines leading to liver injury (Guidotti and Chisari, 2006; Mengshol et al., 2007). Studies in mice demonstrated that ectopic LT overexpression promotes similar chronic inflammation (Drayton et al., 2006). However, whether LT has a significant role in HCV mediated liver injury remains largely unknown.

To determine the role of LT in the pathogenesis of HCV-induced hepatitis, the inventor measured LTα mRNA expression by real-time RT-PCR in liver tissue from human patients chronically infected with hepatitis C virus (FIG. 7). Livers with chronic HCV infection showed significantly increased levels of LTα mRNA expression when compared to uninfected controls (FIG. 7). These data suggest that LT may be involved in the pathogenesis of human HCV induced hepatitis and that blockade of LT by soluble receptors, or other inhibitors of LT expression or activity, will have broad impact on controlling HCV induced hepatitis.

### Materials and Methods

Human samples. Liver tissue samples were collected from chronically infected HCV patients at the time of liver transplantation according to approved procedures at Johns Hopkins School of Medicine. Active chronic hepatitis was confirmed by hematoxylin and eosin staining and AST levels (>50 U/1). All patients were diagnosed chronic hepatitis C virologically and serologically. All healthy donors were negative for HCV, hepatitis B virus (HBV), and human immunodeficiency virus (HIV).

Mice. C57BL/6 male mice 8-10 weeks of age and Fas^{-/-} mice were purchased from The Jackson Laboratory (Jackson Labs Bar harbor, ME). Fas^{-/-} mice were crossed with CD4^{-/-} and CD8^{-/-} mice to generate Fas^{-/-}CD4^{-/-} and Fas^{-/-}CD8^{-/-} double deficient mice, respectively. LTβR^{-/-} mice were kindly provided by K. Pfeffer (Technical University of Munich, Germany) Lck-LIGHT transgenic mice (LIGHT-Tg), LTβ^{-/-} mice were genotyped as described (Wang et al., 2001; Tumanov et al., 2002). All 8-10 weeks aged male mice were on C57BL/6 background and were housed under specific pathogenfree conditions at the University of Chicago. Animal care and use were in accordance with institutional and NIH protocols and guidelines, and all studies were approved by the Animal Care and Use Committee of the University of Chicago.

ConA and antibody treatments. ConA (Vector laboratories) was administered i.v. to the mice at the indicated doses. Sera were collected at the indicated times for measurement of cytokines and liver enzymes. Inhibitors of LT and LTβR signaling: anti-LTβ blocking antibody (BBF6, 100 μg) or soluble LTβR fused to human Fc portion of IgG (LTβR-Ig, 100 μg) were injected i.v. 2 hours prior to ConA injection. The LTβR-Ig used in this study has been previously described. Briefly, cDNA encoding the extracellular domain of murine LTβR was fused with the Fc portion of human IgG, transfected into BHK/VP16 cell, and the supernatant collected.

Induction of hepatitis in Fas^{-/-} mice by administration of anti-CD137. Fas^{-/-} mice were treated with 200 μg agonistic anti-CD137 (2A, rat IgG2a) by i.p. injection. The generation of 2A has been described previously (Wu et al., 1999) and ascites were produced in SCID mice and purified by passage over a protein G-coupled SEPHAROSE® column. Rat IgG was purchased from Sigma-Aldrich and served as a control.

RT-PCR analysis. Total RNA was extracted by RNEASY® mini kit from Qiagen. For cDNA synthesis, RNAs were digested with DNase I and reverse transcribed using random primers with AMV Reverse Transcriptase (Promega) (AMV-RT®). The concentration of the target gene was determined using the comparative C_{T} (threshold cycle number at a cross-point between amplification plot and threshold) method and normalized to GAPDH or HPRT. cDNA were amplified using TAQMAN® PCR MASTER MIX (Applied Biosystems) and run on ABI 7900 cycler (Applied Biosystems). PCR primers and probes used: for human LTα: forward 5′-TGGTGTTGGCCTCACACCT (SEQ ID NO: 1), reverse 5′-CCAGGAGAGAATTGTTGCTC (SEQ ID NO: 2), probe 5′-FAM-CCACAGCACCCTCAAACCTGC-TAMRA (SEQ ID NO: 3); for human GAPDH, forward 5′-GAAGGTGAAGGTCGGAGT (SEQ ID NO: 4), reverse 5′-GAAGATGGTGATGGGATTT (SEQ ID NO: 5), probe 5′-CAAGCTTCCCGTTCTCAGC (SEQ ID NO: 6); for murine MIP-2 (CXCL2): sense 5′-CCACCAACCACCAGGCTACAGGGGC (SEQ ID NO: 7), antisense 5′-AGGCTCCTCCTTTCCAGGTCAGTTAGC (SEQ ID NO: 8); for murine HPRT: forward: 5′-CAGAGGACTAGAACACCTGC (SEQ ID NO: 9), reverse: 5′-GCTGGTGAAAAGGACCTCT (SEQ ID NO: 10); for murine KC (CXCL1): forward 5′-CCACCCGCTCGCTTCTC (SEQ ID NO: 11), reverse 5′-CACTGACAGCGC AGCTCATT (SEQ ID NO: 12); for murine MIP-1α(CCL3): forward 5′-CCTTGCTGTTCTTCTCTGTACCATG (SEQ ID NO: 13), reverse 5′-GCAATCAGTTCCAGGTGAGTGATG (SEQ ID NO: 14).

Histology and TUNEL labeling. Tissues were fixed in 10% buffered formalin and processed either for routine hemotoxylin and eosin staining or TUNEL and immunohistochemical studies. Hematoxylin and eosin staining on sections of embedded tissues was performed according to standard procedure in the University of Chicago Pathology Histology Laboratory. TUNEL staining was performed on paraffin-embedded, formalin-fixed tissue using the APOPTAG® PLUS PEROXIDASE IN SITU APOPTOSIS DETECTION KIT (Chemicon International) according to the manufacturer's directions. Anti-Gr-1 (RB6-8C5 clone) (BD Biosciences) and anti-Myeloperoxidase (MPO) (NeoMarkers) antibody staining was performed on paraffin-embedded, formalin-fixed tissue.

Transaminase activity and cytokines analyses. Blood was collected by retro-orbital puncture, following IACUC approved procedures. Plasma alanine aminotransferase (ALT) and aspartate aminotransferase (AST) activities were determined using a REFLOTRON® PLUS CHEMISTRY ANALYZER according to the manufacturer's procedure (Roche Diagnostics Corp.). Concentrations of cytokines in sera were determined by Cytokine Bead Assay (BD Biosciences) following the manufacturer's recommendations.

Flow cytometry analysis. Intrahepatic leukocytes were purified from the liver by pressing the liver through a steel sieve (Sigma, 190 μm) into PBS, centrifuged at 800 g for 5 min with the resulting pellet suspended in a 35% PERCOLL®-PBS-heparin (100 U/ml) solution and centrifuged at 800 g for 20 min at room temperature. The pellet of mononuclear cells was cleared of RBC with a 5-min osmotic lysis (0.15 M NH₄Cl, 1 mM KHCO₃, 0.1 mM Na₂EDTA, pH 7.3) and washed twice in PBS. Lymphocytes were stained with antibodies (BD Biosciences) and analyzed by flow cytometry (FACSCanto, BD Biosciences).

Statistical analysis. Data are expressed as means±SD. Statistical significance was determined by a two-tailed Student's t-test (*P<0.05, **P<0.01, ***P<0.001), NS-not significant (P>0.05).

Abbreviations used: LT, lymphotoxin; LIGHT, T-cell-restricted ligand, homologous to lymphotoxin, exhibits inducible expression, competes with herpesvirus glycoprotein D for herpesvirus entry mediator on T-cells (TNFSF14); ALT, alanine aminotransferase

### PUBLICATIONS

These publications are incorporated by reference to the extent they relate materials and methods disclosed herein.
Ajuebor, M. N., C. M. Hogaboam, T. Le, A. E. Proudfoot, and M. G. Swain. 2004. CCL3/MIP-1alpha is pro-inflammatory in murine T cell-mediated hepatitis by recruiting CCR1-expressing CD4(+) T cells to the liver. Eur J Immunol 34:2907-2918.
Altemeier, W. A., X. Zhu, W. R. Berrington, J. M. Harlan, and W. C. Liles. 2007. Fas (CD95) induces macrophage proinflammatory chemokine production via a MyD88-dependent, caspase-independent pathway. J Leukoc Biol 82:721-728.
An, M. M., K. X. Fan, Y. B. Cao, H. Shen, J. D. Zhang, L. Lu, P. H. Gao, and Y. Y. Jiang. 2006. Lymphtoxin beta receptor-Ig protects from T-cell-mediated liver injury in mice through blocking LIGHT/HVEM signaling. Biol Pharm Bull 29:2025-2030.
Anand, S., P. Wang, K. Yoshimura, I. H. Choi, A. Hilliard, Y. H. Chen, C. R. Wang, R. Schulick, A. S. Flies, D. B. Flies, G. Zhu, Y. Xu, D. M. Pardoll, L. Chen, and K. Tamada. 2006. Essential role of TNF family molecule LIGHT as a cytokine in the pathogenesis of hepatitis. J Clin Invest 116:1045-1051.
Anders, R. A., S. K. Subudhi, J. Wang, K. Pfeffer, and Y. X. Fu. 2005. Contribution of the lymphotoxin beta receptor to liver regeneration. J.Immunol. 175:1295-1300.
Bajt, M. L., A. Farhood, and H. Jaeschke. 2001. Effects of CXC chemokines on neutrophil activation and sequestration in hepatic vasculature. Am J Physiol Gastrointest Liver Physiol 281:G1188-1195.
Bonder, C. S., M. N. Ajuebor, L. D. Zbytnuik, P. Kubes, and M. G. Swain. 2004. Essential role for neutrophil recruitment to the liver in concanavalin A-induced hepatitis. J Immunol 172:45-53.
Browning, J. L., and L. E. French. 2002. Visualization of lymphotoxin-beta and lymphotoxin-beta receptor expression in mouse embryos. J.Immunol. 168:5079-5087.
Cao, Q., R. Batey, G. Pang, A. Russell, and R. Clancy. 1998. IL-6, IFN-gamma and TNF-alpha production by liver-associated T cells and acute liver injury in rats administered concanavalin A. Immunol Cell Biol 76:542-549.
Chen, M. C., M. J. Hwang, Y. C. Chou, W. H. Chen, G. Cheng, H. Nakano, T. Y. Luh, S. C. Mai, and S. L. Hsieh. 2003. The role of apoptosis signal-regulating kinase 1 in lymphotoxin-beta receptor-mediated cell death. J Biol Chem 278:16073-16081.
Costelli, P., P. Aoki, B. Zingaro, N. Carbo, P. Reffo, F. J. Lopez-Soriano, G. Bonelli, J. M. Argiles, and F. M. Baccino. 2003. Mice lacking TNFalpha receptors 1 and 2 are resistant to death and fulminant liver injury induced by agonistic anti-Fas antibody. Cell Death Differ 10:997-1004.
Desbarats, J., and M. K. Newell. 2000. Fas engagement accelerates liver regeneration after partial hepatectomy. Nat Med 6:920-923.
Dong, Z., H. Wei, R. Sun, and Z. Tian. 2007. The roles of innate immune cells in liver injury and regeneration. Cell Mol Immunol 4:241-252.
Drayton, D. L., S. Liao, R. H. Mounzer, and N. H. Ruddle. 2006. Lymphoid organ development: from ontogeny to neogenesis. Nat Immunol 7:344-353.
Faouzi, S., B. E. Burckhardt, J. C. Hanson, C. B. Campe, L. W. Schrum, R. A. Rippe, and J. J. Maher. 2001. Anti-Fas induces hepatic chemokines and promotes inflammation by an NF-kappa B-independent, caspase-3-dependent pathway. J Biol Chem 276:49077-49082.
Franki, A. S., K. Van Beneden, P. Dewint, K. J. Hammond, S. Lambrecht, G. Leclercq, M. Kronenberg, D. Deforce, and D. Elewaut. 2006. A unique lymphotoxin {alpha}beta-dependent pathway regulates thymic emigration of V{alpha}14 invariant natural killer T cells. Proc Natl Acad Sci USA 103:9160-9165.
Fu, Y. X., and D. D. Chaplin. 1999. Development and maturation of secondary lymphoid tissues. Annu Rev Immunol 17:399-433.
Grove, M., and M. Plumb. 1993. C/EBP, NF-kappa B, and c-Ets family members and transcriptional regulation of the cell-specific and inducible macrophage inflammatory protein 1 alpha immediate-early gene. Mol Cell Biol 13:5276-5289.
Guidotti, L. G., and F. V. Chisari. 2006 Immunobiology and pathogenesis of viral hepatitis. Annu Rev Pathol 1:23-61.
Hatada, S., T. Ohta, Y. Shiratsuchi, M. Hatano, and Y. Kobayashi. 2005. A novel accessory role of neutrophils in concanavalin A-induced hepatitis. Cell Immunol 233:23-29.
He, J. Q., S. K. Saha, J. R. Kang, B. Zarnegar, and G. Cheng. 2007. Specificity of TRAF3 in its negative regulation of the noncanonical NF-kappa B pathway. J Biol Chem 282:3688-3694.
Hehlgans, T., P. Muller, P. Stopfer, and D. N. Mannel. 2003. Activation of the lymphotoxin-beta receptor induces NFkappaB-dependent interleukin-6 and MIP-2 secretion in mouse fibrosarcoma cells. Eur Cytokine Netw 14:103-107.
Huard, J., H. Lochmuller, G. Acsadi, A. Jani, B. Massie, and G. Karpati. 1995. The route of administration is a major determinant of the transduction efficiency of rat tissues by adenoviral recombinants. Gene Ther 2:107-115.
Kaneko, Y., M. Harada, T. Kawano, M. Yamashita, Y. Shibata, F. Gejyo, T. Nakayama, and M. Taniguchi. 2000. Augmentation of Valpha14 NKT cell-mediated cytotoxicity by interleukin 4 in an autocrine mechanism resulting in the development of concanavalin A-induced hepatitis. J Exp Med 191:105-114.
Kim, Y. S., S. A. Nedospasov, and Z. G. Liu. 2005. TRAF2 plays a key, nonredundant role in LIGHT-lymphotoxin beta receptor signaling. Mol Cell Biol 25:2130.
Kuai, J., E. Nickbarg, J. Wooters, Y. Qiu, J. Wang, and L. L. Lin. 2003. Endogenous association of TRAF2, TRAF3, cIAP1, and Smac with lymphotoxin beta receptor reveals a novel mechanism of apoptosis. J Biol Chem 278:14363-14369.
Kusters, S., F. Gantner, G. Kunstle, and G. Tiegs. 1996. Interferon gamma plays a critical role in T cell-dependent liver injury in mice initiated by concanavalin A. Gastroenterology 111:462-471.
Lukashev, M., D. Lepage, C. Wilson, V. Bailly, E. Garber, A. Lukashin, A. Ngam-Ek, W. Zeng, N. Allaire, S. Perrin, X. Xu, K. Szeliga, K. Wortham, R. Kelly, C. Bottiglio, J. Ding, L. Griffith, G. Heaney, E. Silverio, W. Yang, M. Jarpe, S. Fawell, M. Reff, A. Carmillo, K. Miatkowski, J. Amatucci, T. Crowell, H. Prentice, W. Meier, S. M. Violette, F. Mackay, D. Yang, R. Hoffman, and J. L. Browning. 2006. Targeting the Lymphotoxin-{beta} Receptor with Agonist Antibodies as a Potential Cancer Therapy. Cancer Res. 66:9617-9624.
Matsui, H., Y. Hikichi, I. Tsuji, T. Yamada, and Y. Shintani. 2002. LIGHT, a member of the tumor necrosis factor ligand superfamily, prevents tumor necrosis factor-alpha-mediated human primary hepatocyte apoptosis, but not Fas-mediated apoptosis. J Biol Chem 277:50054-50061.
Mengshol, J. A., L. Golden-Mason, and H. R. Rosen. 2007. Mechanisms of Disease: HCV-induced liver injury. Nat Clin Pract Gastroenterol Hepatol 4:622-634.
Nakamura, K., M. Okada, M. Yoneda, S. Takamoto, Y. Nakade, K. Tamori, K. Aso, and I. Makino 2001. Macrophage inflammatory protein-2 induced by TNF-alpha plays a pivotal role in concanavalin A-induced liver injury in mice. J Hepatol 35:217-224.
Niu, L., S. Strahotin, B. Hewes, B. Zhang, Y. Zhang, D. Archer, T. Spencer, D. Dillehay, B. Kwon, L. Chen, A. T. Vella, and R. S. Mittler. 2007. Cytokine-mediated disruption of lymphocyte trafficking, hemopoiesis, and induction of lymphopenia, anemia, and thrombocytopenia in anti-CD137-treated mice. J Immunol 178:4194-4213.
Ponton, A., M. V. Clement, and I. Stamenkovic. 1996. The CD95 (APO-1/Fas) receptor activates NF-kappaB independently of its cytotoxic function. J Biol Chem 271:8991-8995.
Postic, C., M. Shiota, K. D. Niswender, T. L. Jetton, Y. Chen, J. M. Moates, K. D. Shelton, J. Lindner, A. D. Cherrington, and M. A. Magnuson. 1999. Dual roles for glucokinase in glucose homeostasis as determined by liver and pancreatic beta cell-specific gene knock-outs using Cre recombinase. J Biol Chem 274:305.
Ogasawara, J., R. Watanabe-Fukunaga, M. Adachi, A. Matsuzawa, T. Kasugai, Y. Kitamura, N. Itoh, T. Suda, and S. Nagata. 1993. Lethal effect of the anti-Fas antibody in mice. Nature 364:806-809.
Okamoto, S., S. Yokohama, M. Yoneda, M. Haneda, and K. Nakamura. 2005. Macrophage inflammatory protein-1alpha plays a crucial role in concanavalin A-induced liver injury through induction of proinflammatory cytokines in mice. Hepatol Res 32:38-45.
Ramaiah, S. K., and H. Jaeschke. 2007. Role of neutrophils in the pathogenesis of acute inflammatory liver injury. Toxicol Pathol 35:757-766.
Ramos, C. D., K. S. Fernandes, C. Canetti, M. M. Teixeira, J. S. Silva, and F. Q. Cunha. 2006. Neutrophil recruitment in immunized mice depends on MIP-2 inducing the sequential release of MIP-1alpha, TNF-alpha and LTB(4). Eur J Immunol 36:2025-2034.
Rennert, P. D., D. James, F. Mackay, J. L. Browning, and P. S. Hochman 1998. Lymph node genesis is induced by signaling through the lymphotoxin beta receptor. Immunity. 9:71-79.
Rooney, I. A., K. D. Butrovich, A. A. Glass, S. Borboroglu, C. A. Benedict, J. C. Whitbeck, G. H. Cohen, R. J. Eisenberg, and C. F. Ware. 2000. The lymphotoxin-beta receptor is necessary and sufficient for LIGHT-mediated apoptosis of tumor cells. J Biol Chem 275:14307.
Shakhov, A. N., I. G. Lyakhov, A. V. Tumanov, A. V. Rubtsov, L. N. Drutskaya, M. W. Marino, and S. A. Nedospasov. 2000. Gene profiling approach in the analysis of lymphotoxin and TNF deficiencies. J Leukoc Biol 68:151-157.
Seino, K., N. Kayagaki, K. Takeda, K. Fukao, K. Okumura, and H. Yagita. 1997. Contribution of Fas ligand to T cell-mediated hepatic injury in mice. Gastroenterology 113:1315-1322.
Tacke, F., T. Luedde, and C. Trautwein. 2008. Inflammatory Pathways in Liver Homeostasis and Liver Injury. Clin Rev Allergy Immunol
Tiegs, G., J. Hentschel, and A. Wendel. 1992. A T cell-dependent experimental liver injury in mice inducible by concanavalin A. J Clin Invest 90:196-203.
Tiegs, G. 2007. Cellular and cytokine-mediated mechanisms of inflammation and its modulation in immune-mediated liver injury. Z Gastroenterol 45:63-70.
Tumanov, A. V., P. A. Christiansen, and Y.-X. Fu. 2007. The role of lymphotoxin receptor signaling in diseases Curr Mol Med 7:567-578.
Tumanov, A., D. Kuprash, M. Lagarkova, S. Grivennikov, K. Abe, A. Shakhov, L. Drutskaya, C. Stewart, A. Chervonsky, and S. Nedospasov. 2002. Distinct role of surface lymphotoxin expressed by B cells in the organization of secondary lymphoid tissues. Immunity 17:239-250.
Vinay, D. S., B. K. Choi, J. S. Bae, W. Y. Kim, B. M. Gebhardt, and B. S. Kwon. 2004. CD137-deficient mice have reduced NK/NKT cell numbers and function, are resistant to lipopolysaccharide-induced shock syndromes, and have lower IL-4 responses. J Immunol 173:4218-4229.
Wang, J., J. C. Lo, A. Foster, P. Yu, H. M. Chen, Y. Wang, K. Tamada, L. Chen, and Y. X. Fu. 2001. The regulation of T cell homeostasis and autoimmunity by T cell-derived LIGHT. J Clin Invest 108:1771-1780.
Wang, Y., S. K. Subudhi, R. A. Anders, J. Lo, Y. Sun, S. Blink, Y. Wang, J. Wang, X. Liu, K. Mink, D. Degrandi, K. Pfeffer, and Y. X. Fu. 2005. The role of herpesvirus entry mediator as a negative regulator of T cell-mediated responses. J Clin Invest 115:711-717.
Ware, C. F. 2005. Network communications: lymphotoxins, LIGHT, and TNF. Annu Rev Immunol 23:787-819.
Widmer, U., K. R. Manogue, A. Cerami, and B. Sherry. 1993. Genomic cloning and promoter analysis of macrophage inflammatory protein (MIP)-2, MIP-1 alpha, and MIP-1 beta, members of the chemokine superfamily of proinflammatory cytokines. J Immunol 150:4996-5012.
Wilcox, R. A., D. B. Flies, G. Zhu, A. J. Johnson, K. Tamada, A. I. Chapoval, S. E. Strome, L. R. Pease, and L. Chen. 2002. Provision of antigen and CD137 signaling breaks immunological ignorance, promoting regression of poorly immunogenic tumors. J Clin Invest 109:651-659.
Wu, Q., Wang, Y., Wang, J., Hedgeman, E. O., Browning, J., and Fu, Y X. The requirement of membrane lymphotoxin for the presence of dendritic cells in lymphoid tissues. J. Exp. Med. 190:629-38, 1999.22.
Xu, Y., K. Tamada, and L. Chen. 2007. LIGHT-related molecular network in the regulation of innate and adaptive immunity. Immunol Res 37:17-32.
Yu, P., Y. Lee, W. Liu, R. K. Chin, J. Wang, Y. Wang, A. Schietinger, M. Philip, H. Schreiber, and Y. X. Fu. 2004. Priming of naive T cells inside tumors leads to eradication of established tumors. Nat Immunol 5:141-149.

1. A method to reduce liver damage in a mammal, the method comprising administering to the mammal an agent that interferes with the LT or TNF pathways.

2. The method of claim 1 wherein the liver damage is associated with treatment of the mammal by an antibody selected from the group consisting of anti-CD137, anti-CTLA-4, anti-GITR, anti-B7-H1, anti-PD-1, anti-B7-H3 and anti-B7x.

3. A composition comprising an agent that interferes with a component of the LT pathway and a therapeutic agent directed at a disease or condition.

4. The composition of claim 3, wherein the component of the LT pathway is lymphotoxin (LT).

5. The composition of claim 4 wherein the agent that interferes with a component of the LT pathway interferes with LTβR signaling.

6. The composition of claim 3 wherein the agent that interferes with a component of the LT pathway comprises a soluble form of the LTβR or its equivalent.

7. The composition of claim 3 wherein the agent that interferes with a component of the LT pathway comprises the extracellular domain of LTβR fused to a human IgG Fc domain.

8. The composition of claim 3 wherein the agent that interferes with a component of the LT pathway is an antagonistic antibody against LTβR or LT.

9. The composition of claim 3 wherein the disease is selected from the group consisting of an autoimmune disease, viral hepatitis and cancer.

10. A composition comprising an agent that interferes with a component of the TNF pathway and a therapeutic agent directed at a disease or condition.

11. The composition of claim 10 wherein the component of the TNF pathway is TNF.

12. The composition of claim 10 wherein the agent that interferes with a component of the TNF pathway comprises a soluble form of a TNFR or its equivalent.

13. The composition of claim 10 wherein the agent that interferes with a component of the TNF pathway is selected from the group consisting of Infliximab, mouse-human chimeric anti-huTNF mAb, D2E7, fully human anti-huTNF mAb, p75sTNF-RII-Fc, PEG-p55sTNF-RI, p55sTNF-RI-IgG1, CDP571, PEG-linked anti TNF Fab, competitive TNF antagonists, and TNF siRNAs.

14. The composition of claim 10 wherein the disease is selected from the group consisting of an autoimmune disease, viral hepatitis and cancer.

15. The composition of claim 10 wherein the therapeutic agent is an antibody that targets coinhibitory or costimulatory T cell receptors.

16. A pharmaceutical composition to alleviate hepatic injury induced by therapies, the composition comprising an agonistic CD137 antibody and an anti-TNF or anti-LT therapy.

17. The pharmaceutical composition of claim 21 wherein the agonistic CD137 antibody is BMS-663513.

18. The method of claim 1 where the mammal is a human.

19. The method of claim 1 wherein the agent is selected from the group consisting of an antibody and a soluble receptor.
